(19) United States
(12) Patent Application Publication
(10) Pub. No.: US 2011/0183378 A1
(43) Pub. Date: Jul. 28, 2011

(54) NUCLEIC ACID AMPLIFICATION METHOD, NUCLEIC ACID AMPLIFICATION APPARATUS, AND CHIP USED IN NUCLEIC ACID AMPLIFICATION

(75) Inventors: Fumio TAKAGI, Chino (JP); Hiroshi KOEDA, Suwa (JP)

(73) Assignee: SEIKO EPSON CORPORATION, Tokyo (JP)

(21) Appl. No.: 12/956,325

(22) Filed: Nov. 30, 2010

(30) Foreign Application Priority Data
Jan. 25, 2010 (JP) 2010-012880

(51) Int. Cl. C12M 1/00; C12P 19/34; C12M 1/10

(52) U.S. Cl. 435/91.2; 435/289.1

(57) ABSTRACT

A nucleic acid amplification method includes introducing a liquid sample into a first chamber of a chip for use in nucleic acid amplification, the chip including a second chamber containing liquid that has a smaller specific gravity than the liquid sample and is immiscible with the liquid sample, injecting the liquid sample into the second chamber from the first chamber by a centrifugal force, regulating a temperature of an end of the chip, and rotating the chip about a rotation axis at a predetermined speed.

### CROSS REFERENCE TO RELATED APPLICATIONS

This application claims priority to Japanese Patent Application No. 2010-012880, filed Jan. 25, 2010, the entirety of which is hereby incorporated by reference.

### BACKGROUND

1. Technical Field

The present invention relates to a nucleic acid amplification method, a nucleic acid amplification apparatus, and a chip for use in nucleic acid amplification.

2. Related Art

JP-B-4-67957 discloses a Polymerase Chain Reaction (PCR) method, which is widely utilized for researches or clinical tests to examine genes in DNA or RNA, for example. Generally in PCR, a liquid sample that contains a reagent and a sample which may include a target DNA is prepared in a container to be placed in a thermal cycler. The thermal cycler repeatedly raises and lowers the temperature in steps of, for example, 55, 74, and 95 degrees Celsius, to amplify the target DNA. In such PCR, however, regulating the temperature of the liquid sample to a certain temperature takes time, and it is one of the reasons for the difficulty in improving operation efficiency. Attempting to instantly raise or lower the temperature of the liquid sample for faster PCR may cause a thermoelectric element of the thermal cycler to wear out sooner while increasing the power consumption.

JP-A-2009-136250 discloses a method to improve operation efficiency by moving the liquid sample toward or away from the thermoelectric element, which is regulated to a predetermined temperature, to raise or lower the temperature of the liquid sample. However, such temperature regulation method has not yet improved the operation efficiency in temperature regulation for the nucleic acid amplification due to a remaining air bubble disturbing the appropriate temperature gradient inside the container (also referred to as a chip for use in nucleic acid amplification).

### SUMMARY

An advantage of some aspects of the invention is to provide a chip for use in nucleic acid amplification that prevents too much air from remaining inside the chip when the liquid sample is injected, and to provide a nucleic acid amplification method and a nucleic acid amplification apparatus using the chip thereby enabling the time they take for the amplification to be shortened while saving cost and electrical power.

According to an aspect of the invention, a nucleic acid amplification method includes introducing a liquid sample into a first chamber of a chip for use in nucleic acid amplification, the chip including a second chamber containing liquid that has a smaller specific gravity than the liquid sample and is immiscible with the liquid sample, injecting the liquid sample into the second chamber from the first chamber by a centrifugal force, regulating a temperature of an end of the chip, and rotating the chip about a rotation axis at a predetermined speed.

By the above nucleic acid amplification method, the liquid sample is introduced into the first chamber of the chip for use in nucleic acid amplification. The chip includes the second chamber that contains liquid that has a smaller specific gravity than the liquid sample and is immiscible with the liquid sample. The liquid sample is injected into the second chamber from the first chamber by a centrifugal force. Then the temperature of an end of the chip is regulated, and the chip is rotated about the rotation axis at a predetermined speed. The liquid sample is moved from one end to the other end by the gravitational force during the rotation, and thus enabling the liquid sample to be regulated from a temperature range at the one end to a temperature range at the other end.

Because the second chamber is filled beforehand with liquid that has a smaller specific gravity than the liquid sample and is immiscible with the liquid sample, the liquid sample may be injected from the first chamber to the second chamber using a centrifugal force without leaving an undesired mass of air bubble. The temperature gradient of the liquid in the second chamber is maintained without getting distorted by an undesired mass of air bubble, enabling a more efficient regulation of the temperature of the liquid sample.

According to another aspect of the invention, a chip for use in nucleic acid amplification into which a liquid sample is to be introduced includes a first chamber, a second chamber, an injection path that connects the first chamber and the second chamber, and that possesses a maximum width smaller than a minimum width of the second chamber, and liquid in the second chamber, the liquid having a smaller specific gravity than the liquid sample and being immiscible with the liquid sample within a predetermined temperature range.

In the above chip for use in nucleic acid amplification, it is preferable for the injection path to have a maximum width that is smaller than a minimum width of the first chamber. The “maximum width” of the injection path herein indicates a part of the injection path where its width is largest, and the “minimum width” of the first chamber (or the second chamber) indicates a part of the first chamber (or the second chamber) where its width is smallest.

Accordingly, the chip includes the first chamber, the second chamber, the injection path, which connects the first chamber and the second chamber and has a maximum width smaller than the minimum width of the second chamber, and liquid that has a smaller specific gravity than the liquid sample and is immiscible with the liquid sample within a predetermined temperature range. Hence, the liquid sample and liquid are separated once the liquid sample is injected from the first chamber into the second chamber. Separation of the liquid sample from the liquid enables a visual check of the location of the liquid sample in the second chamber. The liquid sample forms a spherical shape while separated from the liquid, and hence the liquid sample injected to the second chamber herein may also be referred to as a “droplet”.

It is preferable that a volume of the liquid is equal to or more than a volume of the second chamber from which a volume of the liquid sample is subtracted, and is equal to or less than a sum of the volume of the second chamber, a volume of the injection path, and a volume of the first chamber from which the volume of the liquid sample is subtracted. Having the preferable volume of the liquid produces less chance of an undesired mass of air bubble left inside the second chamber.

According to another aspect of the invention, a nucleic acid amplification apparatus used with a chip for use in nucleic acid amplification into which a liquid sample is to be introduced, the apparatus including a holder that holds the chip, a rotor that rotates the holder about a rotation axis at a predetermined speed, and a temperature regulator disposed along the rotation axis, wherein the chip includes a first chamber, a second chamber, an injection path that connects the first chamber and the second chamber and has a maximum width smaller than a minimum width of the second chamber, and liquid in the second chamber, the liquid having a smaller specific gravity than the liquid sample and being immiscible with the liquid sample within a predetermined temperature range, and the rotor rotates in a manner that changes a distance between the rotation axis and a lowest point in the second chamber relative to a direction of gravitational force.

It is preferable that the chip for use in nucleic acid amplification utilized with the nucleic acid amplification apparatus also includes a plurality of reaction chambers each of which includes the second chamber and the injection path, and the reaction chambers are connected to the first chamber.

It is further preferable for the nucleic acid amplification apparatus that the temperature regulator includes a first temperature regulator and a second temperature regulator, and a distance between the second temperature regulator and the rotation axis is larger than a distance between the first temperature regulator and the rotation axis.

It is also preferable for the nucleic acid amplification apparatus that a reagent to amplify a target DNA is applied inside the second chamber. The term “target DNA” and “target nucleic acid” is used herein to indicate a nucleic acid to be amplified using the nucleic acid amplification apparatus of the invention.

With the above nucleic acid amplification apparatus, the chip is rotated in a manner that a distance between the rotation axis and the lowest point in the second chamber held by the holder changes, the lowest point being with respect to the direction of gravitational force. Such configuration allows the temperature regulator disposed along the rotation axis to regulate a predetermined part in the chip to a predetermined temperature. The temperature of a liquid sample to be injected into the chip may be regulated more efficiently and hence, the amplification method takes less time while saving power and cost.

According to another aspect of the invention, a chip for use in nucleic acid amplification into which a liquid sample is to be introduced includes a first chamber disposed at center of the chip, and a plurality of reaction chambers each of which includes a second chamber and an injection path connecting the first chamber and the second chamber, in which the reaction chambers are connected to the first chamber and arranged radially around the center.

Using the chip for use in nucleic acid amplification enables different reagents to be applied in each of the plurality of reaction chambers to enable amplification of different target DNAs.

### BRIEF DESCRIPTION OF THE DRAWINGS

The invention will be described with reference to the accompanying drawings, wherein like numbers reference like elements:

FIG. 1A is a perspective view of a nucleic acid amplification apparatus in its entirety according to a first embodiment of the invention;

FIG. 1B is a perspective view of the nucleic acid amplification apparatus in FIG. 1A from which a holding member is removed;

FIG. 1C is a perspective view of the nucleic acid amplification apparatus in FIG. 1B from which a heat insulator is removed;

FIG. 2 is an expanded side view (from the left-hand side of FIGS. 1A to 1C in the angle perpendicular to an extending direction of a rotation axis A) of a first temperature regulator, a second temperature regulator, and a slot for a chip taken out of the nucleic acid amplification apparatus of FIG. 1B;

FIG. 3A is a cross-sectional view of a chip for use in nucleic acid amplification to be placed on the nucleic acid amplification apparatus according to the first embodiment of the invention;

FIG. 3B is a plan view of a first base of the chip for use in nucleic acid amplification;

FIG. 4 is an explanatory drawing of the chip of FIG. 3B in which liquid is filled;

FIG. 5A is an explanatory drawing of the chip of FIG. 3B into which a droplet is introduced;

FIG. 5B is an explanatory drawing of the chip of FIG. 3B into which the droplet has been injected and has moved in the liquid inside the chip;

FIG. 6A is an explanatory drawing of the chip of FIG. 3B in which the droplet is kept at a first temperature;

FIG. 6B is an explanatory drawing of the chip of FIG. 3B in which the droplet is kept at a second temperature;

FIG. 7A is a perspective view of a nucleic acid amplification apparatus in its entirety according to a second embodiment of the invention;

FIG. 7B is a perspective view of the nucleic acid amplification apparatus in FIG. 7A from which a holding member and a second temperature regulator are removed;

FIG. 7C is a perspective view of the nucleic acid amplification apparatus in FIG. 7B from which a measurement port, a heat insulator, and a chip for use in nucleic acid amplification are removed;

FIG. 8 is a plan view of a chip for use in nucleic acid amplification according to the second embodiment of the invention;

FIGS. 9A, 9B, and 9C are cross-sectional views of the chip of FIG. 8 for use in nucleic acid amplification explaining a method to fill the chip with liquid and to introduce a liquid sample (droplet);

FIGS. 10A, 10B, 10C and 10D are plan views of the chip of FIG. 8 for use in nucleic acid amplification explaining a method for nucleic acid amplification; and

FIG. 11 is a plan view of the chip of FIG. 8 for use in nucleic acid amplification according to a modified embodiment.

### DESCRIPTION OF EXEMPLARY EMBODIMENTS

A nucleic acid amplification method, a nucleic acid amplification apparatus, and a chip for use in nucleic acid amplification (hereinafter referred to as a “chip”) according to a first embodiment of the invention will be further described below.

### 1. First Embodiment

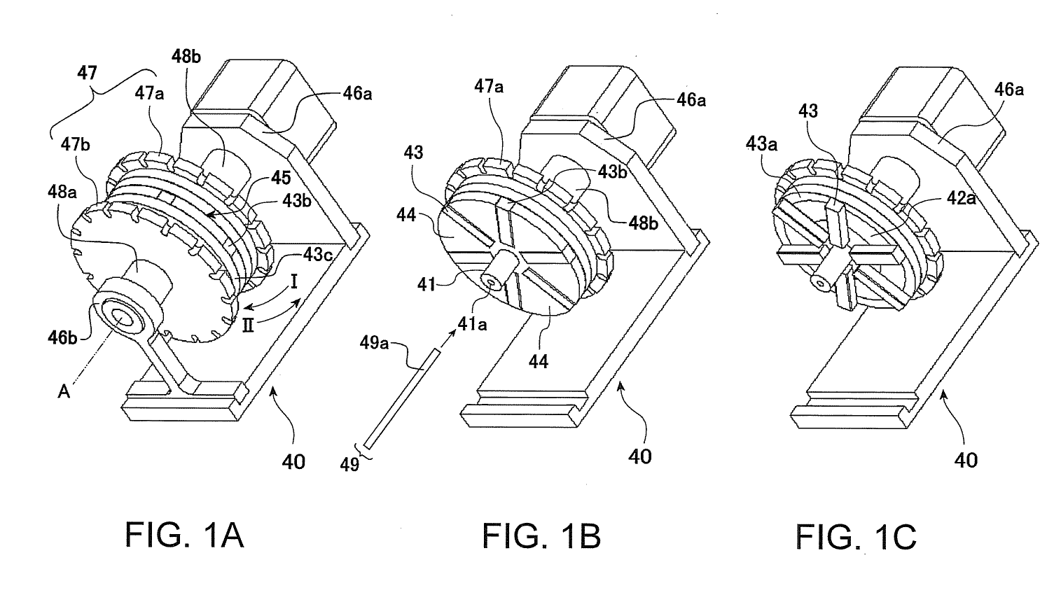

FIGS. 1A, 1B, and 1C are perspective views of a nucleic acid amplification apparatus 40 according to a first embodiment of the invention. FIG. 1A is a perspective view of the nucleic acid amplification apparatus 40 in its entirety. FIG. 1B is a perspective view of the nucleic acid amplification apparatus 40 in FIG. 1A from which a holding member 47b is removed. FIG. 1C is a perspective view of the nucleic acid amplification apparatus 40 in FIG. 1B from which a heat insulator 44 is removed.

### 1.1. Structure of Nucleic Acid Amplification Apparatus

The nucleic acid amplification apparatus 40 according to the first embodiment, as illustrated in FIGS. 1A, 1B, and 1C, includes a temperature regulator (which has a first temperature regulator 49 and a second temperature regulator 45), a holder 47 that holds a chip 100, and a rotor 41 capable of rotating the chip 100 about a rotation axis A.

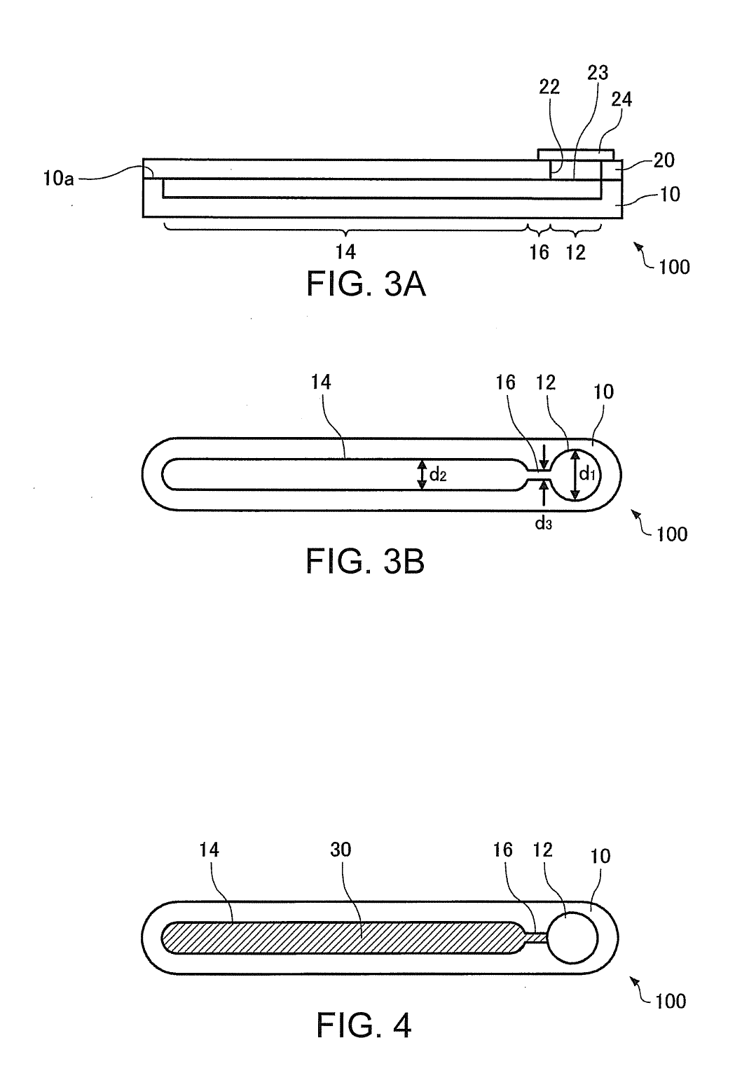

The chip 100, as illustrated in FIGS. 3A and 3B, includes a first chamber 12, a second chamber 14, and an injection path 16 that connects the first chamber 12 and the second chamber 14 disposed on a first surface 10a of a base 10.

The holder 47 holds the chip 100 in a manner that the gravitational force is exerted on the chip 100 in its longitudinal direction as the chip 100 rotates.

The temperature regulator regulates a part of the chip 100 at a predetermined temperature. The first temperature regulator 49 is disposed along the rotation axis A.

The rotor 41 functions in a manner that changes a distance between the rotation axis A and the lowest point in the second chamber 14 with respect to the direction of gravitational force as the chip 100 rotates.

A plurality of slots 43 are disposed on the nucleic acid amplification apparatus 40 in a manner symmetric to the rotation axis A and radially around the rotation axis A. A single slot 43 holds a single chip 100 (not shown in FIGS. 1A to 1C). A heat insulator 44 is disposed between two slots 43 disposed next to each other, as illustrated in FIGS. 1B and 1C, to prevent heat transfer from one slot to the next. The chip 100 of FIGS. 6A and 6B may be positioned in a slot 43 with the first chamber 12 closer to the rotation axis A than the second chamber 14. The chip 100 of FIGS. 6A and 6B may also be positioned in a slot 43 with the second chamber 14 closer to the rotation axis A than the first chamber 12.

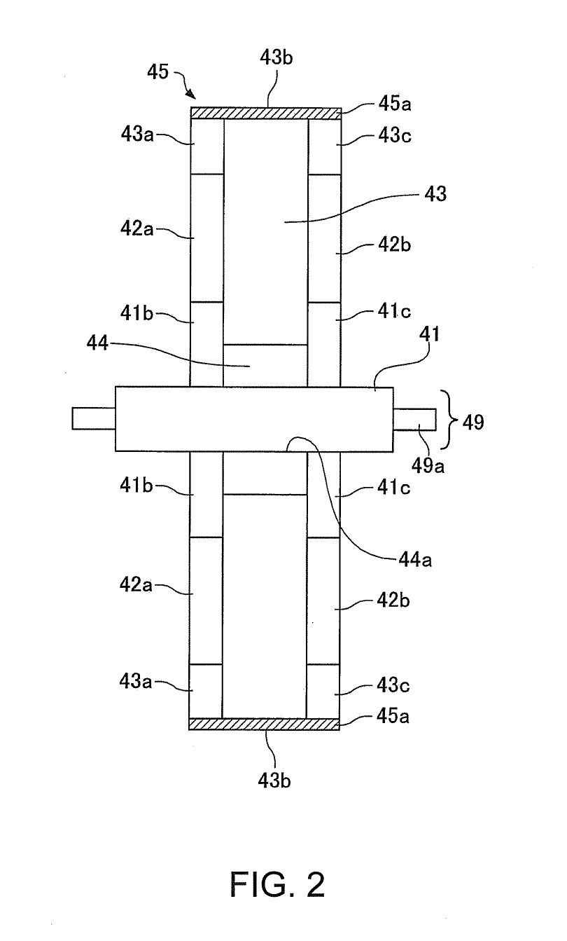

FIG. 2 is an expanded side view (from the left-hand side of FIGS. 1A to 1C in the angle perpendicular to an extending direction of the rotation axis A) of the first temperature regulator 49, the second temperature regulator 45 and the slot 43 taken out of the nucleic acid amplification apparatus of FIG. 1B.

In the nucleic acid amplification apparatus 40 according to the first embodiment, the temperature regulator is configured to include the temperature regulator 49 and the second temperature regulator 45. As illustrated in FIG. 1B, the second temperature regulator 45 is disposed farther from the rotation axis A than is the first temperature regulator 49. The first temperature regulator 49 may be disposed along the rotation axis A as illustrated in FIG. 1B.

The second temperature regulator 45 is disk-shaped as illustrated in FIG. 2. The second temperature regulator 45 has a second heater 45a in the outer circumference.

The rotor 41 rotates the holder 47 about the rotation axis A at a predetermined speed so as to rotate the chip 100. The rotor 41 is made of metal such as aluminum. The heat insulator 44 is disposed surrounding the rotor 41 as illustrated in FIG. 2. The heat insulator 44 is disposed between thermal conductors 41b and 41c. The thermal conductor 41b, a heat insulator 42a, and a thermal conductor 43a form a concentric disk. The thermal conductor 41c, a heat insulator 42b, and a thermal conductor 43c form a concentric disk. The slot 43 and the heat insulator 44 are disposed between the two concentric disks. At least a part of the rotor 41 is disposed inside an opening 44a that penetrates through the thermal conductor 41b, the heat insulator 44, and the thermal conductor 41c. The thermal conductors 41b and 41c are made of metal such as aluminum.

The slot 43 that holds the chip 100 has an optical port (also referred to as a “measurement port”) 43b that optically reads information of the chip 100. The optical port 43b detects concentration of nucleic acid that has been amplified in the second chamber 14. Other devices such as an LED or a CCD sensor (not shown) may be disposed in proximity to the optical port 43b. As such, performing real-time PCR using the nucleic acid amplification apparatus 40 according to the present embodiment allows an optical measurement of the concentration of the amplified nucleic acid in the second chamber 14 as the chip 100 rotates and faces the CCD sensor, enabling the measurement of the amount of PCR product in every cycle.

The thermal conductors 43a and 43c are made of metal such as aluminum and are disposed adjacent to the second heater 45a. The heat from the second heater 45a is transferred to the thermal conductors 43a and 43c to enable a predetermined part of the chip 100 in the slot 43 to be regulated to a predetermined temperature. The second heater 45a may be, for example, a film heater disposed on the outer circumference. The opening 44a penetrates through the thermal conductor 41b, the heat insulator 44, and the thermal conductor 41c.

The first temperature regulator 49 includes a first heater 49a and a first heater holder (an opening in the rotor 41) 41a that holds the first heater 49a, as illustrated in FIG. 1B and FIG. 2. The first heater 49a may be, for example, a bar heater disposed along the rotation axis A and through the opening 44a.

The first heater holder 41a, which is an opening in the rotor 41, is disposed along the rotation axis A. The first heater holder 41a holds the first heater 49a. As such, the first temperature regulator 49 is able to regulate a part of the chip 100 in the slot 43 in proximity to the rotation axis A at a predetermined temperature. More specifically, the first temperature regulator 49 includes the first heater 49a disposed along the rotation axis A to cause a temperature gradation in the chip 100 in such manner that the temperature of a part farther away from the rotation axis A is lower than a temperature of a part closer to the rotation axis A. The first heater holder 41a is disposed in a cylinder-shaped cover denoted 48a and 48b.

For example, when the first chamber 12 of the chip 100 is placed closer to the rotation axis A than the second chamber 14, the first temperature regulator 49 regulates the part in proximity to the rotation axis A to a temperature suitable for thermal denaturation of PCR (for example, 95 degrees Celsius). The second temperature regulator 45 regulates the part away from the rotation axis A to a temperature suitable for annealing and extension of the base sequence (for example, 60 degrees Celsius). Such regulation enables a part of the second chamber 14 of the chip 100 close to the first chamber 12 to be regulated to the temperature for thermal denaturation of PCR (for example, 95 degrees Celsius), and a part of the second chamber 14 away from the first chamber 12 to be regulated to the temperature for annealing and extension of the base sequence (for example, 60 degrees Celsius).

As illustrated in FIG. 1A, the second temperature regulator 45 is disposed in an outer circumference of the slot 43 into which the chip 100 is inserted. Holding members 47a and 47b hold the slot 43 therebetween beyond the outer circumference. The holding members 47a and 47b are supported by supports 46a and 47b via the cover denoted 48a and 48b. The rotor 41 is covered by the cover, denoted 48a and 48b, and both ends of the cover are supported by the supports 46a and 46b. Hence, the holding members 47a and 47b rotate in either of the directions indicated by the arrows I and II in FIG. 1A when the cover, denoted 48a and 48b, is rotated in either of the directions of the arrows I and II. The nucleic acid amplification apparatus 40 according to the present embodiment has a motor disposed external to the support 46b, on which the rotor 41 is disposed, to control the rotation of the rotor 41.

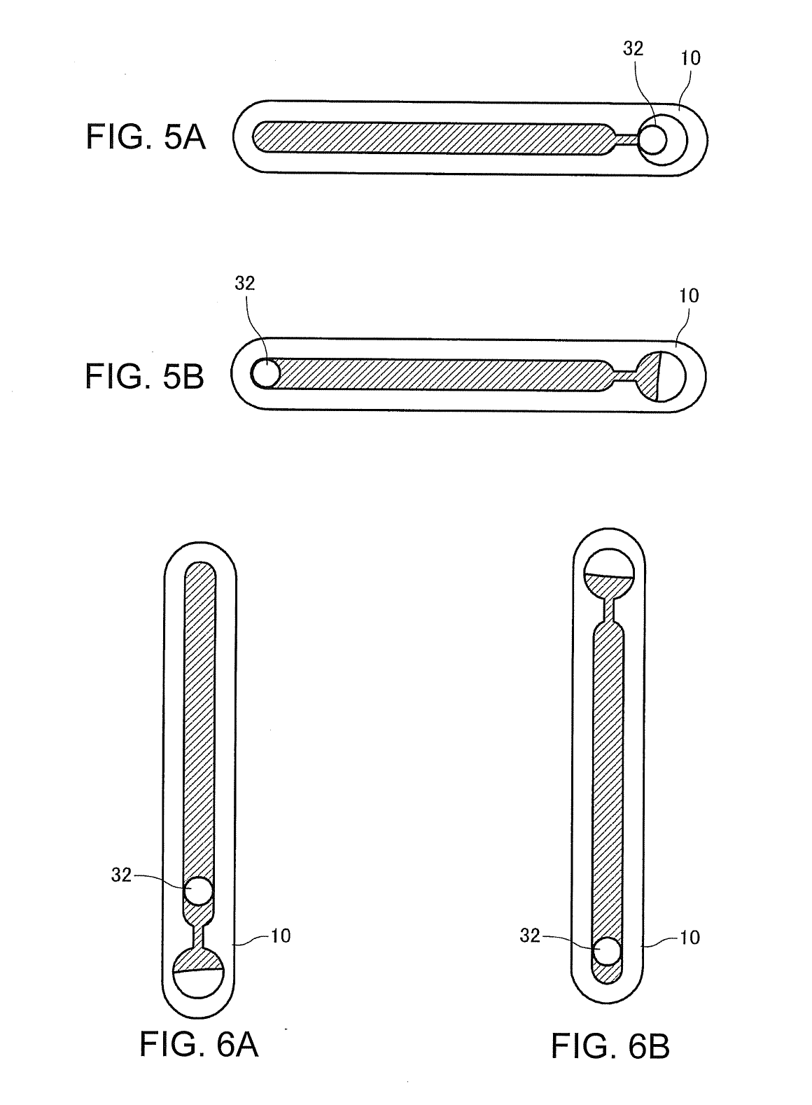

The chip 100 rotates about the rotation axis A along with the rotor 41 rotating about the rotation axis A. The rotor 41 has the holder 47 fixed thereto and hence the holder 47 rotates about the rotation axis A along with the rotor 41 rotating about the rotation axis A. A liquid sample 32 (illustrated in FIG. 5A) introduced into the first chamber 12 of the chip 100 may be injected to the second chamber 14 (illustrated in FIG. 5B) via the injection path 16 by a centrifugal force generated by a centrifuge (not shown). The centrifugation may be performed at 1,000 to 15,000 rpm (rotation per minute) for 0.5 to 5 minutes, for example.

### 1.2. Structure of Chip

The chip 100 to be utilized with the nucleic acid amplification apparatus 40 of the present embodiment is described below. FIG. 3A is a cross-sectional view of the chip 100 to be placed on the nucleic acid amplification apparatus 40 according to the present embodiment. FIG. 3B is a plan view of the first base 10 of the chip 100. Similarly to FIG. 3B, FIGS. 4, 5A, 5B, 6A, and 6B referenced further below also illustrate plan views of the first base 10 of the chip 100.

The chip 100 is a chip for use in nucleic acid amplification into which the liquid sample 32 is to be introduced. As illustrated in FIGS. 3A and 3B, the chip is structured to include the first chamber 12, the second chamber 14, and the injection path 16 that connects the first chamber 12 and the second chamber 14 and has a maximum width d₃ smaller than a minimum width d₂ of the second chamber 14. The chip 100 is constructed of two transparent bases (the first base 10 and a second base 20) as shown in FIG. 3A. The first chamber 12, the second chamber 14, and the injection path 16 are disposed on the first surface 10a of the first base 10. An opening 22 is disposed on the second base 20. The first base 10 and the second base 20 are bonded in a manner that the opening 22 and an introduction port 23 come in contact on the first chamber 12.

Materials to be used for the first base 10 and the second base 20 are not specially specified, although a heat-resistant material with low intrinsic fluorescence is more preferable. An example of such material is resin, such as polycarbonate. Resin is preferred because the material for the first base 10 and the second base 20 needs to be hydrophobic in order for a droplet (liquid sample) 32 to move inside the chip 100 (described in more detail below). As described further below, the second chamber 14 may be filled with liquid 30 by, for example, vacuum-filling or micropipetting, in which the liquid has a smaller specific gravity than the droplet (liquid sample) 32 and is immiscible with the droplet 32 at a predetermined temperature (for example, 45 to 100 degrees Celsius).

The first chamber 12 has the introduction port 23 in which the liquid sample is to be introduced. As described further below, a droplet 32 (illustrated in FIGS. 5A and 5B) initially prepared as the liquid sample for performing nucleic acid amplification is to be introduced into the first chamber 12 from the introduction port 23 of the first chamber 12.

Inside the second chamber 14, nucleic acid amplification of the droplet 32 occurs. As described further below, the second chamber 14 contains liquid 30 (illustrated in FIGS. 5A and 5B). The second chamber 14 may contain a reagent for amplifying a target DNA, for example, applied on an internal surface. The reagent includes a primer or fluorescent probe, for example. The reagent may be applied and dried on an internal surface of the second chamber 14 to stay thereon dried. The reagent may remain in a droplet form in the liquid 30. The reagent applied on the internal surface dissolves into the droplet 32 once the droplet (liquid sample) 32 comes in contact with the reagent. A suitable reagent may be selected depending on a kind of the target DNA. Therefore, when using a plurality of chips 100 with the nucleic acid amplification apparatus 40 of the first embodiment, various kinds of target DNAs may be amplified in the separate chips 100, in which case it is preferable that a suitable reagent is selected for each of the target DNAs.

Examples of nucleic acid subject for nucleic acid amplification may include DNA in samples such as blood, urine, saliva, and spinal fluid, or cDNA which are reverse-transcribed from RNA extracted from the samples mentioned above.

As illustrated in FIG. 3B, the injection path 16 has the maximum width d₃ that is smaller than the minimum width d₂ of the second chamber 14. Such structure effectively prevents a droplet (liquid sample) 32 (illustrated in FIG. 5, described further below) from entering the first chamber 12 from the second chamber 14. It also prevents an air bubble from entering the second chamber 14 from the first chamber 12, and further prevents an air bubble from remaining inside the second chamber 14 when the liquid sample is injected by a centrifugal force.

More specifically, the volume (V=π³/6) of the droplet 32 is preferred to be no less than 0.2 μl and no more than 20 μl in which the diameter of the droplet 32 is denoted “d” mm, given that the droplet 32 is spherical. If the droplet 32 is over 20 μl in volume, it may become unstable and vulnerable. If the droplet 32 is less than 0.2 μl in volume, the droplet 32 may be slowed by the viscosity of the liquid 30.

It is preferred that d₂>d+0.2 mm stands true for the minimum width d₂ of the second chamber 14, considering a smooth movement of the droplet 32, and also that d₂≦2.5 mm stands true considering reducing convective flow inside the second chamber 14.

It is preferred that d₃<d×0.2 mm stands true for the maximum width d₃ of the injection path 16, considering lesser effect of the gravitational force on the droplet 32, and also that d₃≧0.1 mm stands true considering a smooth injection of the droplet 32 through the injection path 16 by the centrifugal force.

### 1.3. Nucleic Acid Amplification Method

A nucleic acid amplification method using the nucleic acid amplification apparatus 40 according to the present embodiment is described hereinafter. The nucleic acid amplification method includes introducing a liquid sample 32 into a first chamber 12 of a chip 100 for use in nucleic acid amplification, the chip 100 including a second chamber 14 containing liquid 30 that has a smaller specific gravity than the liquid sample 32 and is immiscible with the liquid sample 32, injecting the liquid sample 32 into the second chamber 14 from the first chamber 12 by a centrifugal force, regulating the temperature of an end of the chip 100, and rotating the chip 100 about a rotation axis A at a predetermined speed.

By the nucleic acid amplification method of the present embodiment, the chip 100 is rotated about the rotation axis A at a predetermined speed with the liquid sample 32 in the second chamber 14. The rotation causes the liquid sample 32 to move from one end to the other end of the second chamber according to the gravitational force. Moving the liquid sample 32 from the one end, which is in a predetermined temperature range, to the other end, which is in a different temperature range, enables the temperature of the liquid sample 32 to be regulated. Described hereinafter is how the liquid 30 is inserted into the chip 100. FIG. 4 is an explanatory drawing of the chip of FIG. 3B in which the liquid 30 is filled. The second chamber 14 of the chip 100 may be filled by, for example, vacuum-filling or micropipetting the liquid 30. The second chamber 14 receives the liquid 30 from, for example, an inlet disposed thereon (not shown).

The liquid 30 may be oil, for example. Any oil may be used as long as the oil has a smaller specific gravity than water, is immiscible with water, and does not react with reagents or samples used for the nucleic acid amplification. Examples of such oil are mineral oil or silicone oil. Adjusting the viscosity of the liquid 30 enables speed control of the droplet (liquid sample) 32, or in other words, regulation of temperature changes of the droplet 32.

It is preferable, considering removing an air bubble as much as possible, that the volume of the liquid 30 is equal to or more than the amount that fills the volume of the second chamber 14 from which the volume of the liquid sample (droplet) 32 is subtracted, and is equal to or less than the sum of the volume of the second chamber 14, the volume of the injection path 16, and the volume of the first chamber 12 from which the volume of the liquid sample (droplet) 32 is subtracted.

Then the droplet (liquid sample) 32 is introduced from an introduction port 23 into the first chamber 12 by opening a cap 24 of the chip 100, as illustrated in FIG. 5A.

FIG. 5A is an explanatory drawing of the chip 100 of FIG. 3B into which the droplet 32 has been introduced.

The droplet 32 may be introduced into the first chamber 12 by a micropipette. The droplet 32 is a mixture of suitable concentration of, for example, a sample that may include the target DNA, PCR Master Mix, a primer, and a fluorescent probe. A reagent such as a primer or a fluorescent probe may be applied on an internal surface of the second chamber 14, in the case of which the droplet 32 may have no reagent. The suitable amount of the droplet 32 introduced into the chip is described above.

Then the chip 100 is placed on a centrifuge (not shown) with the opening 22 of the first chamber 12 closed (sealed with the cap 24, for example). The chip 100 is positioned in a manner that a distance from the center of the centrifuge to the second chamber 14 is longer than that to the first chamber 12. Any available centrifuge on the market may be used. By using centrifugal force, the droplet 32 is injected from the first chamber 12 towards the second chamber 14. As a result, the droplet 32 is injected in the second chamber 14 through the injection path 16 from the first chamber 12 and moved to an end of the second chamber 14 (the end farther away from the first chamber 12), as illustrated in FIG. 5B. FIG. 5B is an explanatory drawing of the chip 100 of FIG. 3B in which the droplet 32 has been injected and has moved in the liquid 30 inside the chip 100.

The chip 100 is then placed on the nucleic acid amplification apparatus 40 of the present embodiment in a manner that the first chamber 12 is positioned closer to the rotation axis A, and that the second chamber 14 is positioned closer to the measurement port 43b.

The first temperature regulator 49 regulates a part in proximity to the rotation axis A to a first temperature and the second temperature regulator 45 regulates a part away from the rotation axis A to a second temperature that is lower (or higher) than the first temperature. In this case, rotating the chip 100 moves the droplet 32 away from the rotation axis A according to the gravitational force, enabling the temperature of the droplet 32 to be regulated to the second temperature. Further rotating the chip 100 moves the droplet closer to the rotation axis A according to the gravitational force, enabling the temperature of the droplet 32 to be regulated to the first temperature.

For example, when the first temperature is the temperature suitable for thermal denaturation of PCR (for example, 95 degrees Celsius) and the second temperature is the temperature suitable for annealing and extension of the base sequence (for example, 60 degrees Celsius), moving the droplet according to the gravitational force to an appropriate point enables the droplet 32 to be regulated to a predetermined temperature for performing nucleic acid amplification. The nucleic acid amplification apparatus 40 of the present embodiment is positioned such that 0<θ₁<90° stands true for an acute angle (denoted “θ₁”) between the chip 100 and the direction of the gravitational force, because the gravitational force is utilized to move the droplet 32.

More specifically, when rotating the chip 100, positioning the first chamber 12 lower, in other words further down towards the direction of the gravitational force, than the second chamber 14, as illustrated in FIG. 6A, moves the droplet 32 toward the first chamber 12 inside the second chamber 14 according to the gravitational force, due to the specific gravity difference between the liquid 30 and the droplet 32. That enables the droplet 32 to be regulated to a temperature (the first temperature) of the part in proximity to the rotation axis A (this process is referenced below as “Step 1”).

When rotating the chip 100, positioning the second chamber 14 lower, in other words further down towards the direction of the gravitational force, than the first chamber 12, as illustrated in FIG. 6B, moves the droplet 32 away from the first chamber 12 inside the second chamber 14 according to the gravitational force, due to the specific gravity difference between the liquid 30 and the droplet 32. That enables the droplet 32 to be regulated to a temperature (the second temperature lower (or higher) than the first temperature) of the part away from the rotation axis A (this process is referenced below as “Step 2”).

Iterating Steps 1 and 2 enables amplification of the target DNA that may exist in the droplet 32, during which 1 to 20 iterations per minute may be performed.

When the first base 10 made of resin is used to structure the chip 100, a surface of the first base 10 is hydrophobic and hence able to prevent the droplet 32 from sticking onto the first surface 10a of the chip 100. Such structure enables the droplet 32 to smoothly move inside the chip 100.

### 1.4. Features

With the nucleic acid amplification apparatus 40 of the present embodiment, the chip 100 is held inside the slot 43 and the rotor 41 functions to rotate the chip 100 about the rotation axis A. Because the droplet 32 inside the second chamber 14 of the chip 100 has a larger specific gravity than the liquid 30, rotating the chip 100 to change its orientation moves the droplet 32 down inside the second chamber 14 periodically. Iterating the movement to place the droplet 32 at a predetermined location enables the droplet 32 to be regulated to a predetermined temperature (in other words, the temperature according to PCR heat cycles).

Therefore, the nucleic acid amplification apparatus 40 of the present embodiment requires less amount of a liquid sample for nucleic acid amplification, is able to regulate the temperatures more easily, is able to save power consumption, and takes less time for the nucleic acid amplification process, compared to other known PCR methods, such as repeating to raise or lower the temperature of a heating block in which a reaction tube is inserted, or moving the reaction tube between multiple heating blocks.

### 2. Second Embodiment

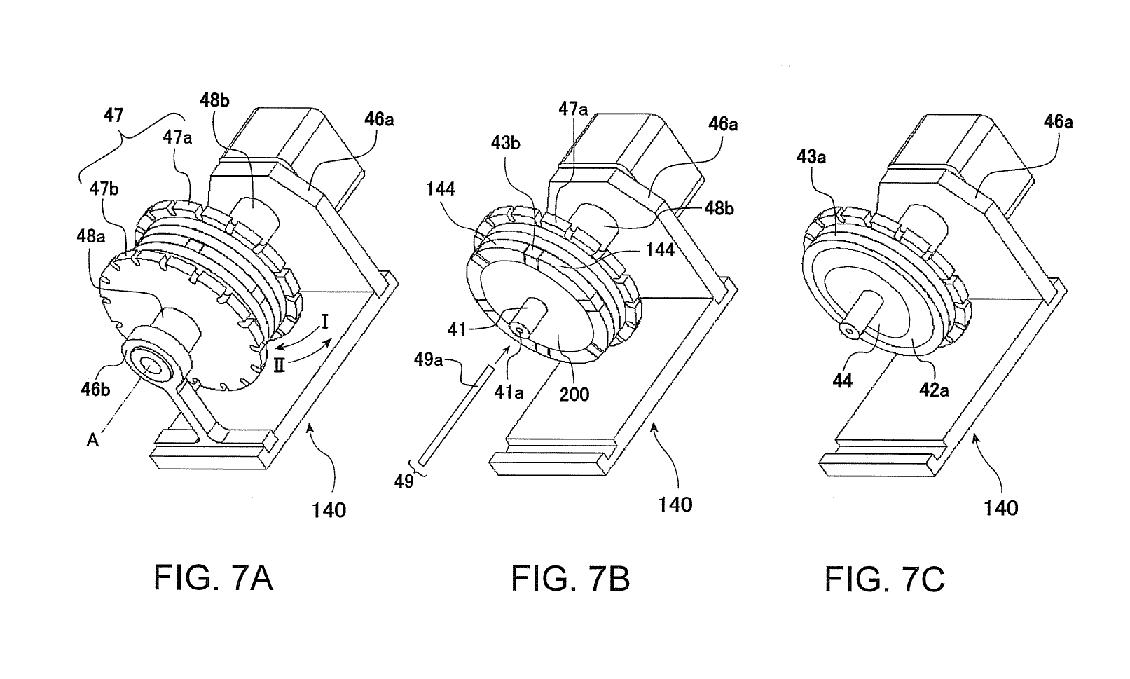

FIGS. 7A to 7C are perspective views of a nucleic acid amplification apparatus 140 according to a second embodiment of the invention. FIG. 7A is a perspective view of the nucleic acid amplification apparatus 140 in its entirety. FIG. 7B is a perspective view of the nucleic acid amplification apparatus 140 in FIG. 7A from which a holding member 47b and a second temperature regulator 45 are removed. FIG. 7C is a perspective view of the nucleic acid amplification apparatus 140 in FIG. 7B from which a measurement port 43b, a heat insulator 144, and a chip 200 for use in nucleic acid amplification are removed.

### 2.1. Structure of Nucleic Acid Amplification Apparatus

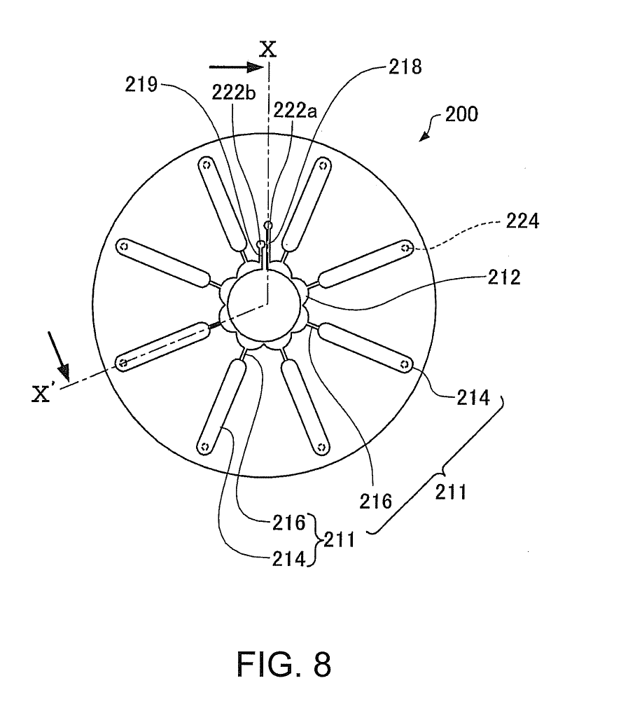

The nucleic acid amplification apparatus 140 according to the present embodiment is different from the nucleic acid amplification apparatus 40 of the first embodiment in a way that the nucleic acid amplification apparatus 140 utilizes the chip 200 (illustrated in FIG. 8) for nucleic acid amplification. The chip 200 includes a first chamber 212, second chambers 214, and injection paths 216 each of which connects the corresponding second chambers 214 to the first chamber 212. The same elements are referenced by the same numerals for both of the nucleic acid amplification apparatuses 40 and 140, and a detailed description of these common elements is thereby omitted.

FIG. 8 is a plan view of the chip 200 to be placed on the nucleic acid amplification apparatus 140 according to the present embodiment.

The chip 200, as illustrated in FIG. 8, is a chip for use in nucleic acid amplification into which a liquid sample 32 is to be introduced. The chip 200 includes the first chamber 212 disposed on the center thereof and reaction chambers 211. Each reaction chamber 211 includes a second chamber 214 and an injection path 216 that connects the second chamber 214 and the first chamber 212.

A plurality of reaction chambers 211 are disposed on the chip 200. The reaction chambers 211 are connected to the first chamber 212 and arranged radially around the center of the chip 200. The first chamber 212, the second chambers 214, and the injection paths 216 are disposed on a first surface 210a of a base (first base 210). Each of the injection paths 216 has a maximum width d₃ that is smaller than a minimum width d₂ of any of the second chambers 214.

Any one of the reaction chambers 211 includes a second chamber 214 connected to the first chamber 212 via an injection path 216. The first chamber 212 includes an inlet 222a into which a liquid sample (droplet) 32 is to be introduced and an outlet 222b from which the air therein is to be discharged. The inlet 222a is connected to the first chamber 212 via a first path 218, and the outlet 222b is connected to the first chamber 212 via a second path 219. The chip 200 includes the first chamber 212, the plurality of second chambers 214 that connect to the first chamber 212 via the injection paths 216, the inlet 222a into which the liquid sample (droplet) 32 is to be introduced, and the outlet 222b from which the air therein is to be discharged. Such structure enables the liquid sample 32 to be provided into all of the second chambers 214 in a single liquid sample injection.

The first base 210 and a second base 220 that structure the chip 200 may be, for example, round as illustrated in FIG. 8. The first chamber 212 is disposed on the center of the first base 210, and the plurality of second chambers 214 are disposed outward from the first chamber 212 radially around the center of the chip 200.

As illustrated in FIG. 8, the first chamber 212 has a larger width in parts facing the second chambers 214 and a smaller width in parts each facing the space in between two of the second chambers 214. Structuring the first chamber 212 in such a manner prevents the liquid sample 32 from going back and forth between the parts with the larger width during the centrifugation, prevents the liquid sample 32 from remaining inside the first chamber 212, and also enables the liquid sample 32 to be distributed substantially equally into the plurality of second chambers 214.

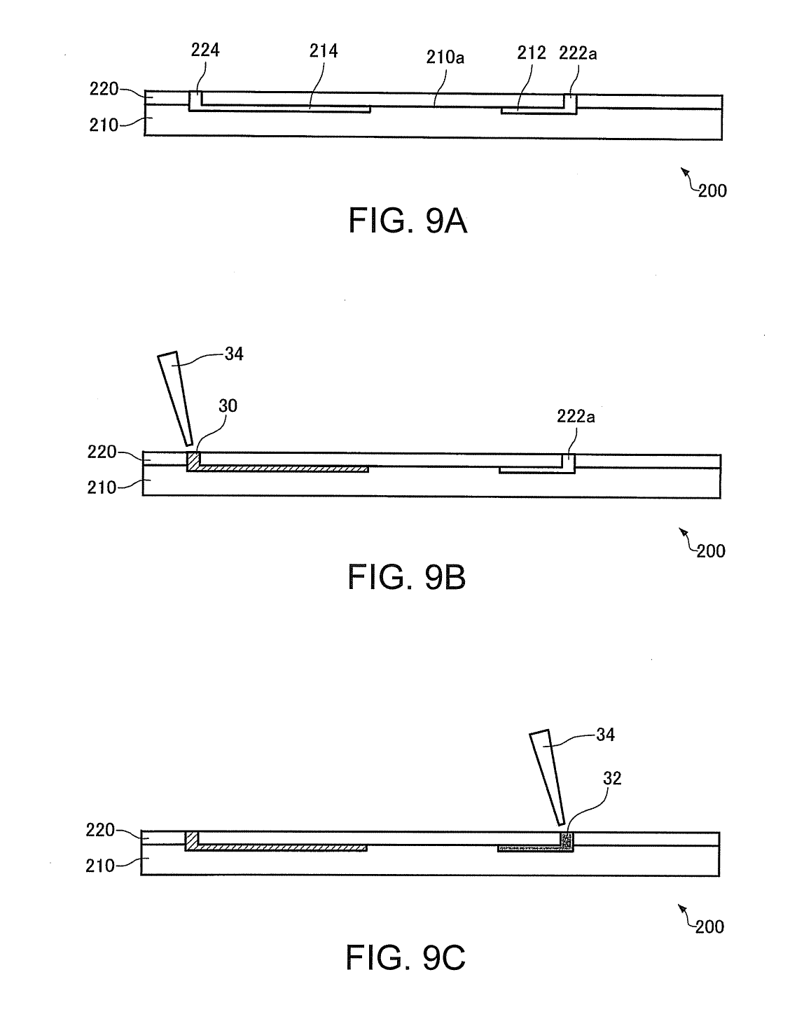

FIGS. 9A, 9B, and 9C are cross-sectional views taken along the line X-X′ of the chip 200 of FIG. 8, explaining a method to fill the chip 200 with liquid 30 and introduce the liquid sample (droplet) 32.

The first chamber 212 and the second chambers 214 are disposed on the first base 210 as illustrated in FIG. 9A. The inlet 222a and an opening 224 are disposed on the second base 220. The inlet 222a is disposed on the first chamber 212, and the opening 224 is disposed on each second chamber 214. The first base 10 and the second base 20 of the first embodiment may be used as the first base 210 and the second base 220.

As illustrated in FIG. 9B, the liquid 30 is inserted into the second chamber 214 from the opening 224 using a pipette 34, for example. Material used for the liquid 30 and a method for inserting the liquid 30 are as described above in relation to the first embodiment. Then the droplet 32 is introduced into the first chamber 212, as illustrated in FIG. 9C, using a pipette 34, for example. Material used for the droplet 32 and a method for introducing the droplet 32 are as described above in relation to the first embodiment.

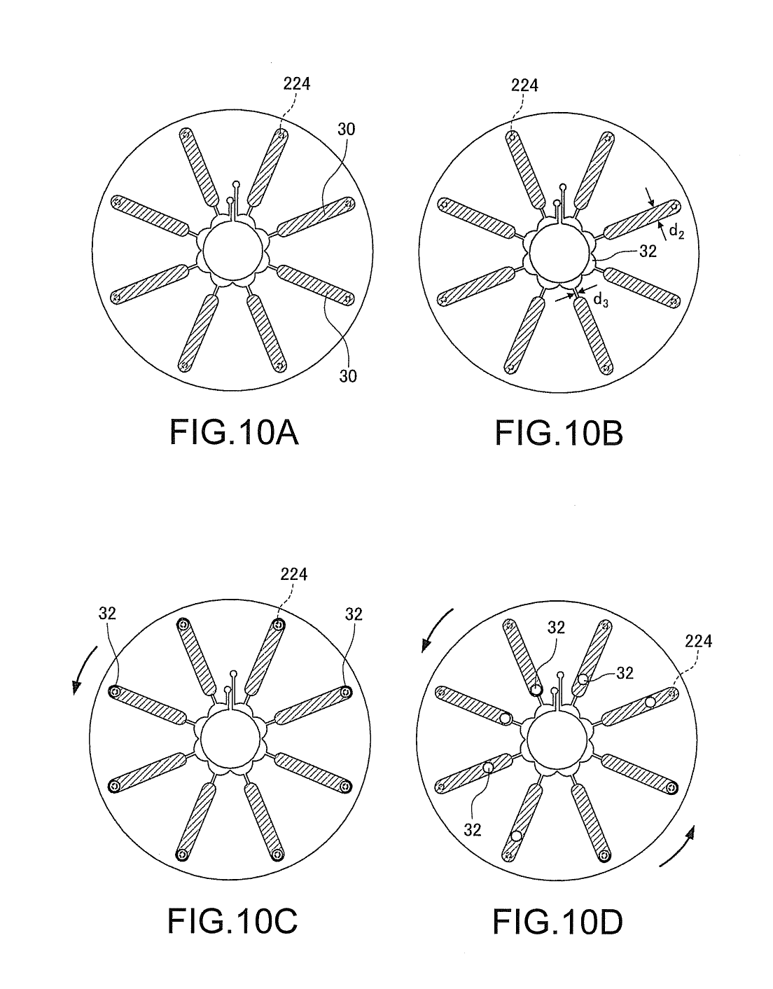

Described below is a method to perform nucleic acid amplification using the chip 200 of FIG. 8. FIG. 10A, 10B, 10C, and 10D are plan views of the chip 200 of FIG. 8, explaining a method for nucleic acid amplification.

First, the liquid 30 is inserted into the second chamber 214 in a manner illustrated in FIG. 9B, to prepare the chip as illustrated in FIG. 10A. Then the liquid sample 32 is introduced into the first chamber 212 in a manner illustrated in FIG. 9C, to prepare the chip as illustrated in FIG. 10B. Centrifuging the chip 200 placed on a centrifuge (not shown) injects, by its centrifugal force, the liquid sample 32 from the first chamber 212 into the second chambers 214 via the injection paths 216, as illustrated in FIG. 10C. The liquid sample injected into the second chambers 214, hereafter referred to as the droplet 32, is further moved within the second chambers 214 to positions away from the rotation axis A close to the outer circumference of the chip 200. The centrifugation may be performed at 1,000 to 15,000 rpm for 0.5 to 5 minutes, for example. Similarly to the first embodiment, any available centrifuge on the market may be used.

Then, as illustrated in FIG. 10D, the chip 200 is placed on the nucleic acid amplification apparatus 140 of the present embodiment and the chip 200 is rotated by the rotor 41 so as to move the droplet 32 closer to the rotation axis A within the second chambers 214 according to the gravitational force. While the droplet 32 remains close to the rotation axis A, the droplet 32 is kept in proximity to the rotation axis A to be regulated to the first temperature as illustrated in the upper-left second chambers 214 of FIG. 10D. (This process is referenced below as “Step 3”.)

Then the chip 200 is rotated by the rotor 41 again after a predetermined time period to move the droplet 32 away from the rotation axis A (as illustrated in FIG. 10C for example) according to the gravitational force within the second chambers 214. Keeping the droplet 32 in locations away from the rotation axis A within the second chambers 214 enables the droplet 32 to be regulated at the second temperature, as illustrated in the lower-right second chambers 214 of FIG. 10D. (This process is referenced below as “Step 4”.)

Iterating Steps 3 and 4 enables amplification of the target DNA that may exist in the droplet 32. The chip 200 may be rotated 1 to 20 rounds per minute in Steps 3 and 4, and the iteration of Steps 3 and 4 may be performed 20 to 60 times. In Steps 3 and 4, the nucleic acid amplification apparatus 140 of the present embodiment is positioned such that 0<θ₂<90° stands true for an acute angle (denoted “θ₂”) between the chip 200 and the direction of the gravitational force, because the gravitational force is utilized to move the droplet 32.

### 2.2. Modified Embodiment

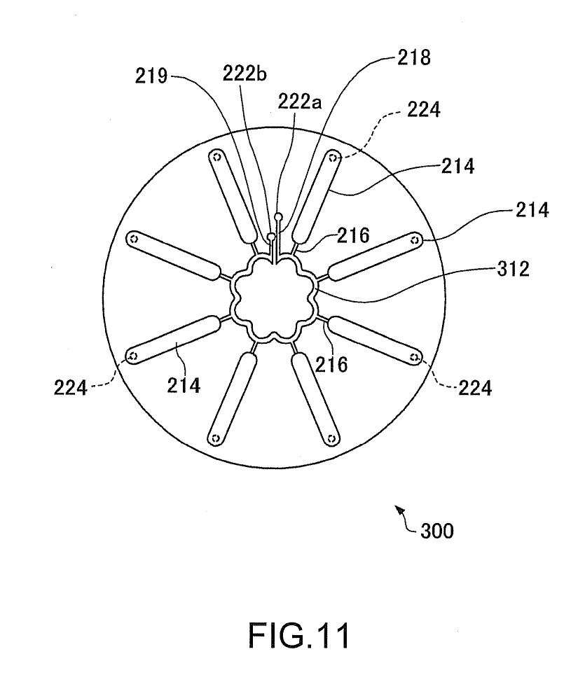

FIG. 11 is a plan view of a chip 300 that is a modification of the chip 200 of FIG. 8. The chip 300 in FIG. 11 has the same structure as the chip 200 of FIG. 8, except the shape of the first chamber 212. The elements with the same or similar functionality as the chip 200 are referenced by the same numerals, and a detailed description of these common elements is thereby omitted.

The chip 300, as illustrated in FIG. 11, has a first chamber 312 that has a substantially uniform width and is wave-shaped. The first chamber 312 is connected to each of the second chambers 214 via the injection paths 216, which connect each of the second chambers 214 to a corresponding outward peak of the waves. The first chamber 312 thus shaped enables the droplet 32 to be easily injected into each of the second chambers 214 from the first chamber 312.

### 2.3. Features

The nucleic acid amplification apparatus 140 according to the present embodiment has the same function and effect as the nucleic acid amplification apparatus 40 of the first embodiment. Further, with the nucleic acid amplification apparatus 140 of the present embodiment, the first chamber 212 (312) of the chip 200 (300) is connected to the plurality of second chambers 214, enabling the liquid sample 32 to be provided into all of the second chambers 214 in a single liquid sample injection.

Preferred embodiments for the present invention are described above. It should be noted that the scope of the invention includes a structure that is substantially the same (for example, its function, way, and result are substantially the same, or its objective and its result are substantially the same as the invention). The scope of the invention also includes a replaceable structure that is immaterial to the invention. The scope of the invention further includes a structure that brings about the same functionality and effect, or that achieves the same objective. The scope of the invention also includes any known structure that may be added to the structure described in the preferred embodiments.

What is claimed is:

1. A nucleic acid amplification method comprising:
introducing a liquid sample into a first chamber of a chip for use in nucleic acid amplification, the chip including a second chamber containing liquid that has a smaller specific gravity than the liquid sample and is immiscible with the liquid sample;
injecting the liquid sample into the second chamber from the first chamber by a centrifugal force;
regulating a temperature of an end of the chip; and
rotating the chip about a rotation axis at a predetermined speed.

2. A chip for use in nucleic acid amplification into which a liquid sample is to be introduced, the chip comprising:
a first chamber;
a second chamber;
an injection path that connects the first chamber and the second chamber, and that possesses a maximum width smaller than a minimum width of the second chamber; and
liquid in the second chamber, the liquid having a smaller specific gravity than the liquid sample and being immiscible with the liquid sample within a predetermined temperature range.

3. The chip for use in nucleic acid amplification according to claim 2, wherein
a volume of the liquid is equal to or more than a volume of the second chamber from which a volume of the liquid sample is subtracted, and
the volume of the liquid is equal to or less than a sum of the volume of the second chamber, a volume of the injection path, and a volume of the first chamber from which the volume of the liquid sample is subtracted.

4. A nucleic acid amplification apparatus used with a chip for use in nucleic acid amplification into which a liquid sample is to be introduced, the apparatus comprising:
a holder that holds the chip, the chip including:
a first chamber,
a second chamber,
an injection path that connects the first chamber and the second chamber and has a maximum width smaller than a minimum width of the second chamber, and
liquid in the second chamber, the liquid including a smaller specific gravity than the liquid sample and being immiscible with the liquid sample within a predetermined temperature range,
a rotor that rotates the holder about a rotation axis at a predetermined speed so as to rotate the chip, and
a temperature regulator disposed along the rotation axis, wherein
the rotor rotates in a manner that changes a distance between the rotation axis and a lowest point in the second chamber relative to a direction of gravitational force.

5. The nucleic acid amplification apparatus according to claim 4, wherein
the chip for use in nucleic acid amplification further comprises
a plurality of reaction chambers each of which includes the second chamber and the injection path, and
the reaction chambers are connected to the first chamber.

6. The nucleic acid amplification apparatus according to claim 4, wherein
the temperature regulator includes a first temperature regulator and a second temperature regulator, and
a distance between the second temperature regulator and the rotation axis is larger than a distance between the first temperature regulator and the rotation axis.

7. The nucleic acid amplification apparatus according to claim 4, wherein
a reagent to amplify a target nucleic acid is applied inside the second chamber.
